(19) United States
(12) Patent Application Publication
(10) Pub. No.: US 2013/0110000 A1
(43) Pub. Date: May 2, 2013

(54) Dual Diameter Introducer Guide Wire

(71) Applicants: Stephen J. Tully, Newark, DE (US); Mark Eberhardt, Elverson, PA (US)

(72) Inventors: Stephen J. Tully, Newark, DE (US); Mark Eberhardt, Elverson, PA (US)

(73) Assignee: Terumo Medical Corporation, Somerset, NJ (US)

(21) Appl. No.: 13/285,984

(22) Filed: Oct. 31, 2011

(51) Int. Cl. A61M 25/09

(52) U.S. Cl. 600/585

(57) ABSTRACT

The present invention is directed to a dual diameter guide wire that is steerable inside the lumen of a patient, and provides greater ability to access small areas such as the interior of a needle, or the interior of a patient's lumen such as small blood vessels, thereby improving access to a patient's body during interventional procedures. The present invention is also directed to medical devices for interventional applications, methods of accessing the interior of a lumen of a patient's body and methods of preparing a guide wire.

### BACKGROUND OF THE INVENTION

The present invention is related to the field of guide wires used for insertion and engaging of medical devices within the body of a patient.

Navigating devices through the interior lumens and passageways of a patient's body is a typical requirement in the art of interventional and surgical procedures. It is typical for a physician to insert a guide wire into a lumen of a patient's body such as a blood vessel or a bile duct, as part of the insertion of a medical device such as a catheter. Insertion of guide wires requires precise and detailed handling, and guide wires are typically very narrow in order to slide easily within the interior of a needle, as well as to accomplish access to a site of interest and accurate placement of medical devices with minimal pain to the patient. However, when an introducer guide wire is slid through a needle and inserted into the lumen, its distal area often has the tendency to get kinked; for example, when meeting with resistance from either the interior of the needle, tissues in the body such as the inner wall of a blood vessel, or with scarred or calcified regions. This may happen in association with trans-radial (TRI) or micro access (MAK) systems.

In a situation where the guide wire gets kinked after insertion into the patient's lumen, the physician often has no choice but to pull out the entire guide wire and start over with the procedure. This can be inconvenient, time-consuming and dangerous. Additionally, in such a situation the guide wire and the patient's lumen may need to be checked for damage after being pulled out. If there is damage done to the guide wire, it must be replaced with a new one; if there is damage done to the patient, the procedure may have to be halted.

In the case where a guide wire kinks during a procedure, less damage may be done if the guide wire has a small diameter. Thus, attempts have been made to manufacture small guide wires in the hope of minimizing likelihood and extent of damage; however, smaller guide wires are harder to control, and thus may prove to be even more likely to kink than larger guide wires. Thus, these types of modifications often lead to a tradeoff between the physician's ability to control the guide wire and the guide wire's strength in moving through both the interior of the needle during placement, and through the patient's lumen. The ideal guide wire should be small enough to be able to maneuver easily through small spaces in the interior of a needle and within the patient's lumen, but also substantial and stiff enough for the physician to handle and guide easily.

Thus, a need exists for improved introducer systems with guide wires that provide precise access to small spaces within a patient's body as well as through the narrow interior of needles, but at the same time provide flexibility, controllability and a lesser likelihood of kinking during interventional procedures.

### SUMMARY OF THE INVENTION

In certain embodiments, the present invention is directed to a guide wire having a proximal portion and a distal portion, the guide wire comprising:
(a) a core wire having a proximal portion with a diameter of about 0.014 to about 0.021 inches, and a distal portion having a diameter of about 0.002 to about 0.008 inches, and terminating at a distal tip; and
(b) a spring disposed on the distal portion of the core wire, the spring comprising a winding wire coiled around the distal portion of the core wire.

In other embodiments, the present invention is directed to a guide wire having a proximal portion and a distal portion, the proximal portion having a diameter in the range of about 0.0195 to about 0.021 inches, and the distal portion having a diameter in the range of about 0.008 to about 0.018 inches;
wherein the guide wire comprises a core wire, and distal portion of the guide wire comprises a radiopaque winding wire coiled around the core wire, the radiopaque winding wire being attached to a portion of the core wire by soldering, brazing, welding or adhesive.

In other embodiments, the present invention is directed to a medical device for an interventional application, the medical device comprising:
(a) a guide wire having a proximal end and a distal end, the proximal end having a diameter in the range of about 0.018 to about 0.021 inches, the distal end having a diameter in the range of about 0.008 to about 0.015 inches, the diameter range of the distal end including a winding wire coiled around the distal end; and
(b) a needle, catheter or introducer sheath.

In other embodiments, the present invention is directed to a method of accessing the interior of a lumen of a patient's body, the method comprising the steps of:
(a) inserting a needle into the interior of a lumen of a patient's body, wherein the needle has a guide wire disposed therein, the guide wire having a proximal end and a distal end, the proximal end having a diameter in the range of about 0.018 to about 0.021 inches, and the distal end having a diameter of about 0.008 to about 0.015 inches, and wherein the guide wire comprises a core wire having a proximal end and a distal end, and a winding wire coiled around the distal end;
(b) advancing the guide wire along the interior of the lumen to a point of interest in the patient's body.

In other embodiments, the present invention is directed to a method of preparing a guide wire, the method comprising the steps of:
(a) obtaining a core wire having a proximal portion and a distal portion, the proximal portion having a proximal diameter and the distal portion having a distal diameter that is smaller than the proximal diameter;
(b) removing material from the distal portion of the core wire to taper the core wire such that the distal diameter is less than the proximal diameter; and
(c) sliding a spring onto the distal portion of the tapered core wire, the spring comprising a coiled winding wire.

### BRIEF DESCRIPTION OF THE DRAWINGS

FIG. 1 shows a guide wire in accordance with certain embodiments of the invention.

FIG. 2 shows a guide wire in accordance with certain other embodiments of the invention.

### DETAILED DESCRIPTION OF THE INVENTION

The embodiments of the present invention address numerous needs in the art—among them, the desirability of providing for more clearance for guide wires to enter or retract the lumen of a patient, even when slightly kinked, thus providing for smoother entrance into the body of a patient and less likelihood of complications associated with interventional procedures.

The guide wires of the present invention provide the advantage of controllability by the user along with flexibility and accuracy in accessing the interior of the body of a patient. It has herein been found that the optimal configuration is one wherein the guide wire is tapered from its proximal end (which the physician will manipulate manually) to its distal end (which is the portion first inserted into the interior of the patient's body). While attempts have been made to provide tapered guide wires, there are currently no known guide wires in the art that are configured such that the interior core wire itself is tapered at its distal portion where the winding wire is coiled around it, and where the winding wire optionally further tapers as it is wound in a direction toward the distal portion of the tapered core wire, and that exhibit the unique advantages of the guide wires of the present invention, as discussed herein. Because, among other reasons, the guide wires of the present invention comprise both tapered core wires and winding wires that are wound in a manner to provide further tapering, the present invention is directed to the novel and unique development of guide wires that exhibit superior properties of controllability and flexibility in this regard.

In keeping with the customary units in the art, the length of the guide wires of the present invention and their components will be expressed in centimeters (cm) while the diameters will be expressed in inches (in).

The guide wires of the present invention are, in certain embodiments, compatible for use with a 21 gauge metal needle transradial (TRI) or micro access (MAK) needle, but of course can be scaled up (to larger diameter needles) or down (to smaller diameter needles) depending on the needs of the particular procedure. Such guide wire systems often have a spring on their distal tip, and are typically slid into the barrel of a needle for use in interventional procedures such as catheterization, insertion of stents, angioplasty and other similar intravascular procedures.

In certain embodiments, a guide wire of the present invention has a length of about 20 to about 100 centimeters, or about 70 to about 90 centimeters, about 40 to about 50 centimeters, or about 45 centimeters or about 80 centimeters. The optimal length of the guide wire can depend on the type of interventional procedure it is used for, and the part of the patient's body that is desired to be accessed. In certain embodiments, the doctor may form the tip of the guide wire into a curve, such as a “J” shape, for better steerability before inserting the tip into the patient's lumen.

### Core Wire

The guide wires of the present invention comprise a core wire. In certain embodiments, the core wire comprises stainless steel or nitinol (a metal alloy of nickel and titanium).

An advantage of the core wires of the present invention over those of the prior art is the fact that they are tapered. In certain embodiments, a core wire of the present invention has a proximal portion and a distal portion, and the proximal portion has a diameter of, in various embodiments, about 0.014 to about 0.021 inches, about 0.015 to about 0.018 inches, about 0.018 to about 0.021 inches, about 0.0195 to about 0.021 inches, about 0.015 inches, about 0.018 inches, about 0.0195 inches, about 0.020 inches or about 0.021 inches. This diameter will be constant along a part of the proximal portion of the core wire, until a point at which it will begin to taper, and finally taper to a point on the distal portion of the core wire where the diameter is, in various embodiments, about 0.001 to about 0.008 inches, about 0.002 to about 0.008 inches, about 0.004 to about 0.005 inches, about 0.004 inches or about 0.005 inches. An important feature of the core wires and the guide wires of the present invention is that they taper from their proximal end to their distal end, so that the proximal diameter should always be greater than the distal diameter.

Most preferably, a first taper should begin at a point along the length of the core wire that is about 5 to about 30 centimeters from the distal tip of the core wire. In various embodiments, a first taper begins at a point that is about 10 to about 20 centimeters from the distal tip of the core wire, or about 12 to about 18 centimeters from the distal tip of the core wire. Other embodiments will be more discussed in reference to the Figures.

In certain embodiments, rather than occurring gradually, the taper is more abrupt in that the core wire goes from a first portion having a proximal diameter of about 0.014 to about 0.021 inches, to a second portion having a distal diameter of about 0.001 to about 0.008 inches. In certain embodiments, a core wire of the present invention may taper only once from this proximal diameter range to this distal diameter range, or may taper several times over multiple portions of the length of the core wire. Generally speaking, a taper over fewer different lengths is more desirable for purposes of performance, efficiency and less material used; however, the embodiments of the core wires of the present invention are not necessarily so limited. In some embodiments, a gradual taper is more desirable than a sudden taper, because a gradual taper generally results in a softer and more atraumatic distal tip, which is easier for the physician to maneuver around bends and curves within the blood vessel, duct or other lumens of the patient's body.

### Spring and Winding Wire

The guide wires of the present invention comprise a spring. In certain embodiments, the spring comprises a winding wire, at least a portion of which may, in certain embodiments, be attached to the core wire at a point on the distal portion of the core wire. As used herein, “spring” refers merely to a coiled winding wire.

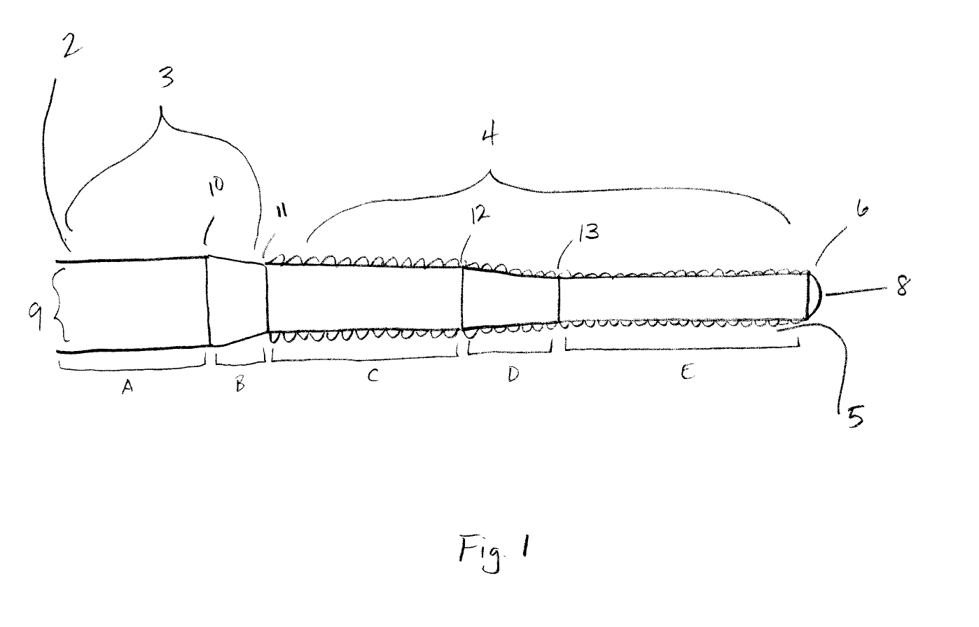

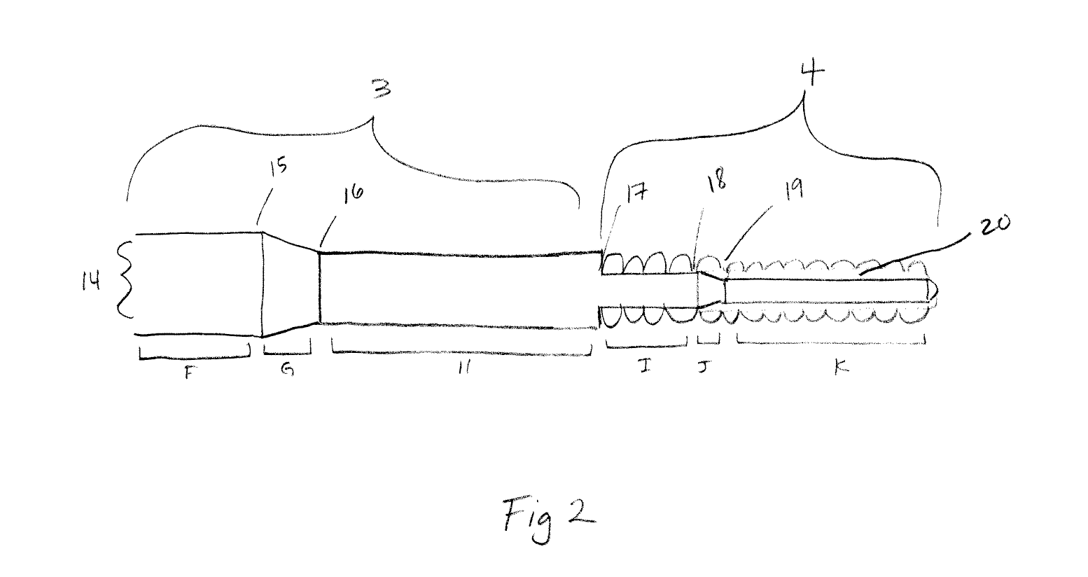

In practice, a guide wire according to the present invention can be shown in, for example, FIG. 1 and FIG. 2. As can be seen in FIG. 1, a guide wire 1 according to the present invention comprises a core wire 2, having a proximal portion 3 and distal portion 4, and a winding wire 5 attached to at least a part of the distal portion 4 of the core wire and wound around the distal portion in a direction toward the distal end 6. In the embodiment shown in FIG. 1, the core wire has a total length of about 40 to about 45 cm. It tapers from the proximal portion to the distal portion—specifically, its proximal diameter 9 is about 0.019 to about 0.021 inches (Section “A). At a first tapering point 10 along the length of the core wire, the diameter begins to taper along a length of about 2 to about 5 cm (Section “B”) down to a point 11 along a length of about 4 to about 5 cm (“Section C”) where the core wire's diameter is about 0.007 to about 0.017 inches. The core wire tapers again starting at another point 12 along a length of about 1.5 to about 2.5 cm (Section “D”) to a point 13 along a length of about 2.5 to about 3 cm (Section “E)” having a core wire diameter of about 0.002 to about 0.004 inches down to the distal end 6, which ends in a distal tip 8. The distal tip should be atraumatic—that is, it should provide maximum smooth entry of the guide wire and minimal pain to the patient. In some embodiments, the distal tip is rounded. In other embodiments, the core wire may be flattened into a “ribbon” as it approaches the distal tip, to provide additional flexibility.

As shown in FIG. 1, the winding wire is coiled around the distal portion of the core wire, and begins its wind at the point 11 shown on the core wire; however, the invention is also not so limited, in that the core wire can be attached at any point along the length of the core wire between the proximal portion and the distal portion. In the embodiments of the invention, the winding wire can be coiled around, for example, the proximal portion of the core wire before any taper begins, or further along the distal portion of the core wire after a taper has begun. Further, in some embodiments the winding wire can be wound in such a way that the overall diameter of the guide wire itself remains constant from proximal end to distal end, in spite of the taper of the core wire—that is, the winding wire will be more loosely wound around the distal end of the core wire than the proximal end of the core wire, to compensate for the decreased diameter from the core wire taper. In other embodiments, the winding wire can be wound in such a way that it provides a taper of the guide wire from proximal portion to distal portion—for example, the winding wire may be more tightly wound from the proximal portion of the core wire as it goes in a direction toward the distal portion of the core wire, to follow the taper of the core wire. In other words, the spring formed by the coiled winding wire may in various embodiments be of uniform diameter along its length, or of a tapering diameter along its length.

In the embodiment shown in FIG. 2, the core wire 2 tapers more abruptly—that is, the core wire's proximal diameter as depicted therein 14 is about 0.019 to about 0.021 inches along a length (Section “F”) until a point along the length 15 where a taper begins, and the core wire tapers along a length (Section “G”) to another point 16 where the core wire diameter is about 0.010 to about 0.020 inches (“Section H”). At yet another point 17, an abrupt taper occurs such that the core wire diameter is about 0.008 to about 0.015 inches along a length (Section “I”). At another point 18, another taper occurs along another length (Section “J”) to another point 19, after which the diameter of the core wire is about 0.001 to about 0.008 inches (Section “K”). In the embodiments pictured here, the length of the core wire is in various embodiments, about 35 to about 40 cm, about 36 to about 37 cm, or about 36.5 cm from the proximal point of Section F to the distal point of Section H; and in various embodiments, about 5 to about 10 cm, about 6 to about 8 cm or about 6.5 cm from the proximal point of Section I to the distal point of the core wire.

Thus, in various embodiments, a guide wire of the present invention may have a proximal portion having the same ranges of diameters as those set forth herein regarding the proximal portion of the core wire, since at the guide wire's proximal portion the diameter is merely the diameter of the core wire. In various embodiments, a guide wire of the present invention may have a distal portion (which includes the distal portion of the core wire and the spring) of about 0.008 to about 0.018 inches, about 0.008 to about 0.015 inches, about 0.008 to about 0.012 inches, about 0.012 to about 0.015 inches, about 0.012 inches, about 0.014 inches, about 0.015 inches or about 0.018 inches.

In certain embodiments, the present invention is directed to a method of preparing a guide wire comprising cutting away a portion of the distal portion of the guide wire to provide a taper from the proximal portion of the core wire to the distal portion of the core wire. The methods of the present invention contemplate grinding, blazing, swaging, hammering or extruding the core wire in order to achieve the desired taper(s).

In the embodiment shown in FIG. 2, the core wire is shown as unitary wire (that is, comprising merely a single piece of metal) with a pronounced difference in diameter between its proximal portion and distal portion, as shown at point 7. However, the guide wire may, in certain other embodiments, comprise multiple core wires joined together end to end or in parallel, in a manner that achieves this same change in diameter.

In the embodiments shown in the Figures, the winding wire 5 is coiled around at last part of the distal portion of the core wire. As can be seen in both FIG. 1 and FIG. 2, in certain embodiments the winding wire and core wire both terminate at a distal tip 8 where both are welded and soldered together, brazed, or connected to each other with adhesive such as, e.g., glue or epoxy.

In various embodiments of the present invention, the winding wire is a radiopaque wire. That is, the spring may comprise a winding wire that is made at least in part of a radiopaque material, including but not limited to gold, platinum, palladium, silver, iridium, nickel, titanium, or stainless steel, or any combinations of any of the foregoing. For example, in certain embodiments, the winding wire may comprise an alloy of gold and platinum, or gold, platinum or iridium; or the winding wire may comprise nitinol, which is an alloy of nickel and titanium. Further, in certain embodiments the guide wire is not limited to a single winding wire, but in fact may comprise two or more winding wires on its distal portion. For example, a spring of the present invention may comprise two winding wires intertwined in a helical pattern, or two winding wires coiled together and in parallel, or two winding wires wherein one is radiopaque but the other is not. The spring may comprise a winding wire or plurality of winding wires wound in such a manner that they contribute to the taper in the distal portion of the guide wire.

Among the many advantages of the guide wires of the present invention are their ability to balance flexibility with controllability. While attempts have been made to provide tapered guide wires, these are generally not desirable for several reasons. In particular, the guide wires currently in use do not include a tapering core wire that tapers to the extent of the guide wires of the present invention—in various embodiments herein, down to diameters of about 0.015 inches or less, and even down to diameters that are even smaller than those of the proximal portion of the core wire. It is known in the art that smaller distal diameters tend to render guide wires difficult to control; therefore, those of ordinary skill in the art have tended to halt the extent to which the distal portions of guide wires are approach very small values, for the reason that such small diameters are generally not practical, are essentially not usable, or for other reasons yield poor results in practice. The guide wires of the present invention, in contrast, have been shown to provide all of the advantages of controllability without the expected downsides.

In other embodiments, the present invention is directed to a medical device for an interventional application comprising a guide wire as described in the present disclosure, and a needle, catheter or introducer sheath. These elements may be packaged together in a kit for convenient access and use by a physician in performing an interventional procedure. The guide wires of the present invention are advantageous in that they slide easily through a needle as part of an access kit, with a minimum of kinking and a maximum of control. In other embodiments, the present invention is directed to methods of accessing the interior of a lumen of a patient's body, comprising inserting a needle therein in conjunction with a guide wire as described in the present disclosure, and advancing the guide wire along the interior of the lumen to a point of interest in the patient's body.

In other embodiments, the present invention contemplates methods of preparing a guide wire, the methods comprising the steps of obtaining a core wire as described herein, and removing a layer of the distal portion of the core wire to taper the core wire. By “removing a layer” it is meant that a portion of the core wire may be ground, blazed, swaged or extruded to provide the desired taper. For example, the core wire may be subjected to grinding, which effectively decreases the diameter of the core wire in a desired location along its length. The core wire may be extruded in such a manner that it becomes tapered along its length. Further, in certain embodiments, the most distal portion of the core wire may be flattened into a “ribbon”—that is, its diameter may be reduced to very small values (for example, about 0.001 to about 0.004 inches) in order to further provide the comfort of an atraumatic distal tip. The method of preparing a guide wire contemplated by the present invention may also include a step of coiling a winding wire around the distal portion of the core wire, in such a manner that the guide wire is tapered from the proximal portion to the distal tip. As discussed previously herein, the winding wire may be wound in such a manner that it gradually tightens from the proximal portion of the core wire to the distal portion of the core wire, to effect a taper, all the while being in contact with the core wire as it tapers. Alternatively, the winding wire may tighten in a more nuanced fashion—for example, as it approaches the distal tip, the wire may be wound tightly enough to provide an overall taper to the guide wire, but there can be some space, or “play” 20 (shown in FIG. 2) between the winding wire and the guide wire, which provides some increased flexibility and springiness to the distal portion of the guide wire.

In certain embodiments, the spring comprising the winding wire is manufactured separately from the core wire—that is, it is formed by winding the winding wire around a cylindrical or elongated object, which is then removed, leaving behind the finished spring. The spring may then be slid onto the distal end of the core wire and in some embodiments, at least a portion of the spring may be attached to the core wire as discussed herein (either by soldering, welding, brazing or adhering) to form a guide wire in accordance with the present invention.

All embodiments described herein are illustrative and in no way limit the scope of the invention, and the invention may be embodied in other forms not explicitly described here, without departing from the spirit thereof

What is claimed is:

1. A guide wire having a proximal portion and a distal portion, the guide wire comprising:
(a) a core wire having a proximal portion with a diameter of about 0.014 to about 0.021 inches, and a distal portion having a diameter of about 0.002 to about 0.008 inches, and terminating at a distal tip; and
(b) a spring disposed on the distal portion of the core wire, the spring comprising a winding wire coiled around the distal portion of the core wire.

2. The guide wire of claim 1, wherein the core wire has a length of about 20 to about 100 centimeters.

3. The guide wire of claim 1, wherein the core wire is tapered from a proximal diameter in the range of about 0.014 to about 0.021 inches, to a distal diameter in the range of about 0.002 to about 0.008 inches, wherein the taper begins at a point that is about 5 to about 30 centimeters from the distal tip of the core wire.

4. The guide wire of claim 1, wherein the core wire has a proximal portion with a diameter of about 0.018 to about 0.021 inches.

5. The guide wire of claim 4, wherein the core wire has a proximal portion with a diameter of about 0.0195 to about 0.020 inches.

6. The guide wire of claim 1, wherein the winding wire has a diameter of about 0.002 to about 0.004 inches.

7. The guide wire of claim 1, wherein the guide wire has a proximal portion having a diameter of about 0.018 to about 0.021 inches, and a distal portion having a diameter of about 0.008 to about 0.015 inches.

8. The guide wire of claim 7, wherein the guide wire has a distal portion having a diameter of about 0.008 to about 0.012 inches.

9. The guide wire of claim 7, wherein the guide wire has a distal portion having a diameter of about 0.012 to about 0.015 inches.

10. The guide wire of claim 1, wherein the core wire comprises nitinol or stainless steel.

11. The guide wire of claim 1, wherein the spring comprises a radiopaque winding wire.

12. The guide wire of claim 11, wherein the winding wire comprises platinum, palladium, gold, silver, tungsten, nitinol or stainless steel, or combinations of any of the foregoing.

13. The guide wire of claim 1, wherein at least a portion of the winding wire is soldered, brazed, welded or adhered to the core wire.

14. The guide wire of claim 1, wherein at least a portion of the winding wire is attached to the core wire with adhesive.

15. The guide wire of claim 12, wherein the winding wire comprises an alloy of gold and platinum.

16. A guide wire having a proximal portion and a distal portion, the proximal portion having a diameter in the range of about 0.0195 to about 0.021 inches, and the distal portion having a diameter in the range of about 0.008 to about 0.018 inches;
wherein the guide wire comprises a core wire, and distal portion of the guide wire comprises a radiopaque winding wire coiled around the core wire, the radiopaque winding wire being attached to a portion of the core wire by soldering, brazing, welding or adhesive.

17. The guide wire of claim 16, the proximal portion having a diameter in the range of about 0.0195 to about 0.021 inches, and the distal portion having a diameter in the range of about 0.012 to about 0.015 inches.

18. The guide wire of claim 17, wherein the core wire has a proximal portion and a distal portion, the proximal portion having a diameter in the range of about 0.0195 to about 0.021 inches, and a distal portion having a diameter in the range of about 0.004 to about 0.008 inches.

19. The guide wire of claim 16, wherein the winding wire comprises nitinol or stainless steel.

20. The guide wire of claim 16, wherein the distal portion of the guide wire comprises two winding wires coiled around the core wire, one of the winding wires being a non-radiopaque winding wire.

21. A medical device for an interventional application, the medical device comprising:
(a) a guide wire having a proximal end and a distal end, the proximal end having a diameter in the range of about 0.018 to about 0.021 inches, the distal end having a diameter in the range of about 0.008 to about 0.015 inches, the diameter range of the distal end including a winding wire coiled around the distal end; and
(b) a needle, catheter or introducer sheath.

22. The medical device of claim 21, wherein the distal end of the guide wire has a diameter in the range of about 0.008 to about 0.012 inches.

23. The medical device of claim 21, wherein the distal end of the guide wire has a diameter in the range of about 0.012 to about 0.015 inches.

24. A method of accessing the interior of a lumen of a patient's body, the method comprising the steps of:
(a) inserting a needle into the interior of a lumen of a patient's body, wherein the needle has a guide wire disposed therein, the guide wire having a proximal end and a distal end, the proximal end having a diameter in the range of about 0.018 to about 0.021 inches, and the distal end having a diameter of about 0.008 to about 0.015 inches, and wherein the guide wire comprises a core wire having a proximal end and a distal end, and a winding wire coiled around the distal end;
(b) advancing the guide wire along the interior of the lumen to a point of interest in the patient's body.

25. The method of claim 24, wherein the interior of the lumen of the patient's body is chosen from a blood vessel or a duct.

26. A method of preparing a guide wire, the method comprising the steps of:
(a) obtaining a core wire having a proximal portion and a distal portion, the proximal portion having a proximal diameter and the distal portion having a distal diameter that is smaller than the proximal diameter;
(b) removing material from the distal portion of the core wire to taper the core wire such that the distal diameter is less than the proximal diameter; and
(c) sliding a spring onto the distal portion of the tapered core wire, the spring comprising a coiled winding wire.

27. The method of claim 26, further comprising the step of attaching the winding wire to the distal portion of the core wire with soldering, brazing, welding or adhesive.

28. The method of claim 26, wherein step (b) is accomplished by grinding, blazing, swaging, hammering or extruding at least a portion of the core wire.
